(19) United States
(12) Patent Application Publication
(10) Pub. No.: US 2020/0016592 A1
(43) Pub. Date: Jan. 16, 2020

(54) HIGH-THROUGHPUT MICROFLUIDIC-BASED METHODS FOR RECORDING DEFECATION MOTOR PROGRAM (DMP) EVENTS IN NEMATODES

(71) Applicant: NemaMetrix Inc., Eugene, OR (US)

(72) Inventors: Kathryn McCormick, Eugene, OR (US); Trisha Brock, Eugene, OR (US)

(73) Assignee: NemaMetrix Inc., Eugene, OR (US)

(21) Appl. No.: 16/452,493

(22) Filed: Jun. 25, 2019

Related U.S. Application Data
(60) Provisional application No. 62/689,806, filed on Jun. 25, 2018.

(51) Int. Cl. B01L 3/00; G01N 33/50; G01N 33/52; G01N 21/64; A01K 67/033

(52) U.S. Cl. CPC B01L 3/502707; B01L 3/502715; G01N 33/5085; G01N 33/52; G01N 2021/178; A01K 67/0336; A01K 2227/703; A01K 2267/035; G01N 21/64

(57) ABSTRACT

The present disclosure provides methods and systems for performing, observing, and/or recording defecation motor program (DMP) events using microfluidic devices. The methods may be performed wherein the nematodes ingest fluorescent or color material and are then loaded in a microfluidic chip and stimulated to feed and defecate. DMP events are observed with use of a fluorescent microscope. In other methods, a microfluidic device with two or more electrodes is used to record electrical events of the DMP.

### CROSS-REFERENCE TO RELATED APPLICATIONS

This application claims the benefit of U.S. Provisional Patent Application No. 62/689,806, filed on 25 Jun. 2018, the content of which is incorporated herein by reference in its entirety.

### STATEMENT REGARDING FEDERALLY SPONSORED RESEARCH OR DEVELOPMENT

This invention was made with government support under RGM 119906B awarded by NIH. The government has certain rights in the invention.

### FIELD OF THE INVENTION

This application pertains generally to microfluidic devices and methods thereof for detecting defecation motor program (DMP) events of a nematode.

### BACKGROUND OF THE INVENTION

Rhythmic motor programs are an important class of behavioral output of biological organisms. These motor programs include behaviors such as breathing, walking, swimming, and defecation and are produced by neuronal circuits know as central pattern generators in the absence of sensory or afferent inputs [Marder E, Bucher D. Central pattern generators and the control of rhythmic movements. Current Biology. 2001 Nov. 27; 11(23)]. Central pattern generators provide a model for the interaction of genetics with the neuronal basis of behavior, and rhythmic motor programs provide easily quantifiable outputs to detect molecular perturbation. The defecation motor program (DMP) of Caenorhabditis elegans (C. elegans) is one such behavior and has been used to investigate and uncover the effects of neurotransmitter receptors and channels such as GABA receptors, voltage-activated potassium channels, correlations between motor programs; DMP is an easy readout for forward genetic screens.

The Defecation Motor Program (DMP) cycle consists of a posterior body contraction (pBoC), anterior body contraction (aBoC) and lastly an enteric muscle contraction (EMC). The cycle occurs once every 40-45 seconds in healthy N2 isolates of C. elegans. If food is dilute, the cycle will be longer (once every 50-60 seconds). The cycle is reasonably constant at 19° C.-30° C.

Previous methods for the quantification of defecation behaviors include visualizing excreta as nematodes crawl in a bacterial lawn on agar plates, and monitoring changes in body length that occur with the pBoC and aBoC DMP events. To keep the nematode in the field of view at sufficiently high power to continuously detect defecation events, visual monitoring requires the investigator to re-position the agar plate multiple times during the assay. Moreover, defecation events are typically monitored on solid media such as agar wherein the nematodes have freedom of movement; there is a known coupling between locomotory behavior and defecation motor programs and defecation and other rhythmic motor programs, such as egg-laying, are suppressed in liquid media. These known methods are time consuming and not amenable to automation or high throughput methodologies.

There is a need for improved methods to detect and record events associated with defecation motor program (DMP) of nematodes. The methods disclosed herein overcome the shortcomings of the known methods wherein fluorescent material is used to visualize defecation events in a constrained microfluidic channel that is amenable to both automation and high throughput.

### SUMMARY OF THE INVENTION

Herein are provided systems and methods for performing a nematode defecation motor assay using a microfluidic chip. Methods provided herein provide high throughput means for observing/recording/measuring DMP events of nematodes.

In embodiments, methods comprise inducing at least one nematode to ingest a fluorescent or colored material, introducing the at least one nematode after ingesting the fluorescent or colored material into a microfluidic chip configured to hold the nematode in a microfluidic channel, and placing the microfluidic chip on a stage of a fluorescent microscope and observing contraction and expulsion events of the at least one nematode, whereby the nematode defecation motor assay is performed.

In embodiments, a system comprises, a microfluidic chip comprising at least one channel configured to hold individual nematodes in each channel, at least one nematode comprising ingested fluorescent or colored material and placed in the at least one channel of the microfluidic chip, and, a fluorescent microscope with excitation and emission filters selected for a fluorophore or chromophore of the ingested fluorescent or colored material.

In embodiments, a method of performing a nematode defecation motor assay using a microfluidic device measuring an electrical event of an electrical muscle discharge comprises introducing the nematode into a microfluidic device configured to hold the nematode in a microfluidic channel, wherein the microfluidic device comprises two or more electrodes directly connected to the microfluidic channel, measuring the electrical event of the nematode and, recording the electrical event as an electrical muscle discharge whereby the nematode defecation motor assay is performed. In certain embodiments, the nematode is present in an aqueous buffer solution comprising food.

In embodiments, the present methods use microfluidic device comprising one or more microfluidic channels, each channel configured to hold one nematode in fluid. In embodiments, the microfluidic device comprises a silicone polymer, a thermoplastic polymer, an acrylic polymer, or a polycarbonate polymer, wherein the thermoplastic polymer comprises poly(methyl methacrylate) (PMMA), polycarbonate (PC), polystyrene (PS), polyvinyl chloride (PVC), polyimide (PI), olefin polymers, cyclic olefin copolymer (COC), cyclic olefin polymer (COP), or cyclic block copolymer (CBC). In certain embodiments, the silicone polymer comprises a polydimethylsiloxane (PDMS) elastomer.

Provided herein are methods for performing functional analysis of a genetic variant. The present methods comprise providing a transgenic nematode comprising a heterologous gene, wherein exon coding sequences of the heterologous gene comprises one or more mutations resulting in an amino acid change as compared to a wildtype reference sequence, introducing the transgenic nematode into a microfluidic device configured to hold the nematode in a microfluidic channel, performing a nematode defecation motor assay using a microfluidic device, comparing results of the defecation motor assay to a result from a defecation motor assay of a control nematode to identify a change between results, whereby functional analysis of a genetic variant is performed.

In embodiments, the methods further comprising placing the microfluidic device on a stage of a fluorescent microscope and observing contraction and expulsion events of the transgenic nematode. In embodiments, the transgenic nematode is induced to ingest a fluorescent or colored material with bacterial feed.

In certain embodiments, the microfluidic device comprises two or more electrodes directly connected to the microfluidic channel, and the defection motor assay is performed by measuring the electrical event of the nematode and recording the electrical event as an electrical muscle discharge.

In certain embodiments, the method further comprises introducing a therapeutic agent into the microfluidic device with the transgenic nematode. In embodiments, that method further comprising identifying therapeutic agents that alter the defecation motor phenotype of the transgenic nematode.

In certain embodiments, the methods for performing functional analysis of a genetic variant further comprise analyzing directionality of the change in results to determine mode of action of the genetic variant.

### BRIEF DESCRIPTION OF THE DRAWINGS

The drawings illustrate generally, by way of example, but not by way of limitation, various embodiments disclosed herein.

FIG. 1A to FIG. 1G show fluorescent images of nematodes recording expulsion and intestinal contraction events while in a microfluidic channel of a microfluidic chip. In all frames, the anterior of the nematode is to the left. FIGS. 1A to 1G represent a time series of frames recorded during an expulsion event, comprising a time period of roughly 1 second. Each image is separated in time by 33-130 ms. FIG. 1A, fluorescent material can be seen in the digestive tract of the nematode. FIG. 1B, the fluorescent material has moved toward the posterior of the nematode, forming a bolus near the anus. FIG. 1C, the fluorescent bolus is moving through the anus. FIG. 1D, the material has been ejected from the nematode. FIG. 1E, the material is moving in the direction of fluid flow, away from the nematode. FIG. 1F, the material is flowing further away from the nematode and dispersing in the medium. FIG. 1G, the fluorescent material had dispersed and has left the field of view.

FIGS. 2A and 2B show an electrical voltage signal recording during the execution of the defecation motor program (DMP). An image of the nematode, posterior to the left, is directly above a voltage recording. The black vertical line indicates the voltage signal recorded at the time corresponding to the depicted frame, wherein FIG. 2A shows measurement prior to the expulsion event and FIG. 2B was measured during the expulsion event. The contraction of the posterior of the animal temporally corresponds to a large voltage fluctuation, roughly shaped like the letter “M”, whereas the small voltage fluctuations present before and after the expulsion event are electrical events from pharyngeal pumping during feeding. The difference in electrical events between feeding and expulsion are easily distinguished as shown in FIGS. 2A and 2B.

FIGS. 3A to 3C show data from the nematode NMX97, which is a knockout of the homolog of the human gene KCNQ2, kqt-3, compared with the nematode wildtype N2. FIG. 3A is a bar graph showing intervals between successive defecation events in seconds for the two nematode strains. FIG. 3B shows a time course of defecation events for multiple individual nematodes of the N2 strain. Each line represents one measured nematode. The interval between the 1st and the 2nd defecation event is labeled “1” on the X axis, and the number of seconds in the interval is represented on the Y axis. FIG. 3C shows a time course of defecation events for multiple individual nematodes of the NMX97 strain. Each line represents one measured nematode. The interval between the 1st and the 2nd defecation event is labeled “1” on the X axis, and the number of seconds in the interval is represented on the Y axis. The knockout of the kqt-3 gene results in a disruption of the peristaltic rhythm and an increase in the interval between successive defecation events. The defecation events were measured as an electrical event and demonstrate that the defection motor phenotype, as measured in a microfluidic device, can distinguish nematodes the functional impact of genetic manipulations.

### DETAILED DESCRIPTION OF THE INVENTION

### Introduction

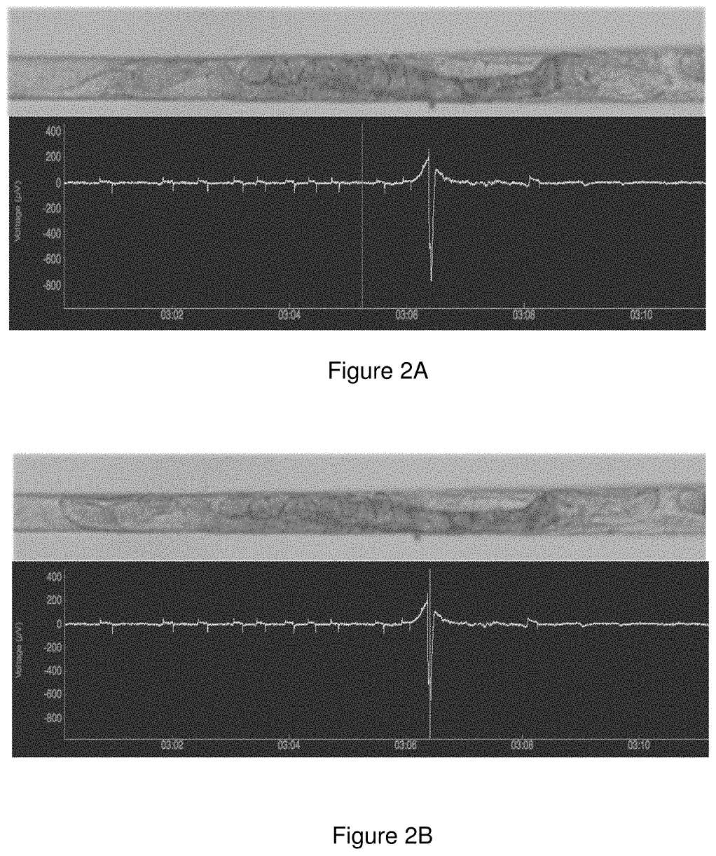

Disclosed herein are methods for performing a nematode defecation motor assay using a microfluidic chip, also referred to herein as a microfluidic device. The methods disclosed herein utilize a microfluidic device, which is amendable to high throughput use, for observing, measuring and recording defection motor phenotype. In certain embodiments, the methods utilize fluorescence wherein nematodes are fed a fluorescent material, such as food or bacteria comprising a fluorophore, wherein the microfluidic device comprising the nematodes is utilized with a microscope (fluorescent or brightfield) to image defection events. See Example 1. In certain other embodiments, the defection motor program (DMP) is measured as an electrical event, representing an electrical muscle discharge, wherein the microfluidic device comprises at least two electrodes directly connected to a microfluidic (recording) channel. See Example 2 and FIG. 2.

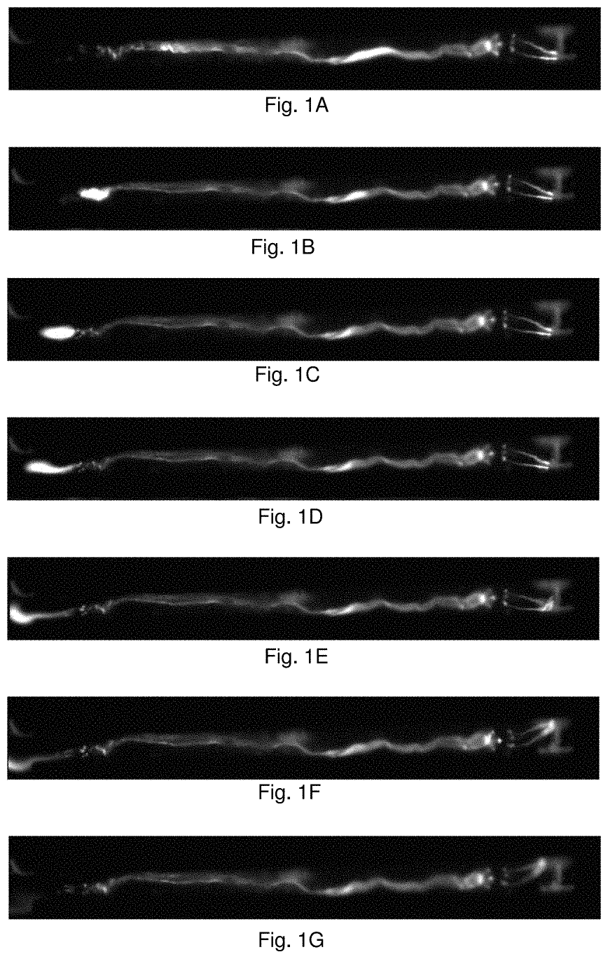

In certain embodiments, the methods involve feeding nematodes fluorescent material to induce fluorescent excretions, placing the nematode in a constrained microfluidic environment, stimulating the nematode to feed and defecate, and monitoring the nematodes visually for expulsion events. See Example 1 and FIG. 1.

In embodiments, the methods comprise inducing at least one nematode to ingest a fluorescent or colored material, after ingesting the fluorescent or colored material, introducing the at least one nematode into a microfluidic chip configured to hold the nematode in a microfluidic channel, and placing the microfluidic chip on a stage of a fluorescent microscope and observing contraction and expulsion events of the at least one nematode, whereby the nematode defecation motor assay is performed.

In certain other embodiments, the methods involve placing the nematode in a constrained microfluidic environment, stimulating the worm to feed and defecate, and monitoring the animal's electrical events for expulsion events. DMP events are distinguished from the electrical events caused by pharyngeal pumping (Electropharyngeogram or EPG) by several characteristics. Primary among these is the waveform. The EPG is characterized by an excitatory “E” peak (in the positive direction) followed by a period of quiescence and then a relaxation “R” peak (in the negative direction) with an interval of roughly 200 ms between the E and the R. The overall waveform resembles an EKG waveform in shape. The DMP waveform overall resembles an “M” in shape, with a positive peak followed by a deep negative peak followed by another positive peak. The amplitude of the voltage signal is another distinguishing feature. The amplitude of the DMP waveform is roughly ˜3 times greater than the EPG waveform produced by the same animal. See FIG. 2.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the terms “Caenorhabditis elegans” or “C. elegans” refer to a free-living transparent nematode, about 1 mm in length, which lives in temperate soil environments. The basic anatomy of C. elegans includes a mouth, pharynx, intestine, gonad, and collagenous cuticle.

As used herein, the term “fluidic device” refers to a device that utilizes the flow of fluid to distribute substances and/or organisms (such as substances dissolved in a fluid and/or substances or organisms suspended in a fluid). A fluidic device can be of any dimension, so long as its dimensions are suitable to accommodate the size of substances or organisms included or suspended in the fluid. In embodiments, a device is a microfluidic device that exploits the properties of fluid flow that arise at length scales in the sub-millimeter range. One such property is laminar flow. In some examples, a microfluidic device has a channel or chamber with at least one dimension of 300 microns or less. In other examples, two dimensions are 300 microns or less. Some microfluidic devices are fabricated in glass whereas others are fabricated in a bio-compatible silicone or thermoplastic polymer by replica molding. The latter are referred to as soft-lithography microfluidic devices. The term “microfluidic device” is sometimes used as a synonym for the more general term “microfabricated device,” which refers to an object that may or may not exploit the properties of fluid flow at the sub-millimeter scale.

As used herein, the term “nematode” refers to an organism that is a member of the phylum Nematoda, commonly referred to as roundworms. Nematodes include free-living species (such as the soil nematode C. elegans) and parasitic species. Species parasitic on humans include ascarids, filarias, hookworms, pinworms, and whipworms. It is estimated that more than two billion people worldwide are infected with at least one nematode species. Parasitic nematodes also infect companion animals and livestock, including dogs and cats (e.g., Dirofilaria immitis; heartworm), pigs (Trichinella spiralis), and sheep (e.g., Haemonchus contortus). There are also nematode species which are parasitic on insects and plants.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. As used herein, “clinical variant” is a disease gene that comprises one or more amino acid changes as compared to wild type and is thus a mutant gene.

A “normal” or “wild type” nucleic acid, nucleotide sequence, polypeptide or amino acid sequence refers to a naturally occurring or endogenous nucleic acid, nucleotide sequence, polypeptide or amino acid sequence that has not undergone a change. As used herein, the wild type sequence may be a disease gene, but does not comprise a mutation leading to a pathogenic phenotype. It is understood there is a distinction between a wild type disease gene (e.g. those without a mutation leading to a pathogenic phenotype and may be an allele reflective of a “normal” heterogenous population) and clinical variants that comprise one or more mutations of those disease genes and that may have a pathogenic phenotype. In embodiments, the normal gene or wild type gene may be the most prevalent allele of the gene in a heterogenous population.

“Partially-functional” as used herein describes a protein that is encoded by a mutant gene and has less biological activity than a functional protein but more than a non-functional protein. In embodiments, function is determined via one or more phenotypic assays wherein a phenotypic profile for the mutant (disease) gene may be generated.

As used herein, the term “percent sequence identity” or “percent identity” refers to the percentage of identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence

As used herein, the term “percent sequence similarity” or “percent similarity” refers to the percentage of near-identical nucleotides in a linear polynucleotide of a reference (“query”) polynucleotide molecule (or its complementary strand) as compared to a test (“subject”) polynucleotide molecule (or its complementary strand) when the two sequences are optimally aligned. In some embodiments, “percent similarity” can refer to the percentage of near-identical amino acids in an amino acid sequence. Near-identical amino acids are residues with similar biophysical properties (e.g., the hydrophobic leucine and isoleucine, or the negatively-charged aspartic acid and glutamic acid).

As used herein, the term “polynucleotide” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA as DNA construct, mRNA, and anti-sense RNA, any of which can be single stranded or double stranded. The terms “polynucleotide,” “nucleotide sequence” “nucleic acid,” “nucleic acid molecule,” and “oligonucleotide” are also used interchangeably herein to refer to a heteropolymer of nucleotides. Except as otherwise indicated, nucleic acid molecules and/or polynucleotides provided herein are presented herein in the 5′ to 3′ direction, from left to right and are represented using the standard code for representing the nucleotide characters as set forth in the U.S. sequence rules, 37 CFR § 1.821-1.825 and the World Intellectual Property Organization (WIPO) Standard ST.25.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%), 98%), 99%), or 100% as compared to a control. In embodiments, the reduction in the context of a heterogenous gene or clinical variant thereof, is measured and/or determined via phenotypic assay to assess function of the expressed gene.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” and grammatical variations thereof in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%>nucleotide or amino acid residue identity, when compared and aligned for maximum correspondence, as measured using one of the following sequence comparison algorithms or by visual inspection. In particular embodiments, substantial identity can refer to two or more sequences or subsequences that have at least about 70%, at least about 75%, at least about 80%, at least about 85%, at least about 90%, at least about 95, 96, 96, 97, 98, or 99% identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Subject” and “patient” as used herein interchangeably refers to any vertebrate, including, but is not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. The subject or patient may be undergoing other forms of treatment. In embodiments, the patient is a human wherein a clinical variant is a sequence of a disease gene from the patient.

“Target gene” as used herein refers to any nucleotide sequence encoding a known or putative gene product. As used herein the target gene may be the chimeric heterologous gene, either in normal or wild type form, or as a clinical variant, or the host animal ortholog of the heterologous gene. The target gene may be a mutated gene involved in a genetic disease, also referred to herein as a clinical variant.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” with respect to a peptide or polypeptide that differs in one or more amino acid sequence by the insertion, deletion, or conservative substitution of amino acids as compared to a normal or wild type sequence. The variant may further exhibit a phenotype that is quantitatively distinguished from a phenotype of the normal or wild type expressed gene. In embodiments, clinical variant refers to a disease gene with one or more amino acid changes as compared to the normal or wild type disease gene.

All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Microfluidic Device

The instant system comprises a microfluidic device and a nematode. In embodiments, any microfluidic device can be used wherein the device comprises at least one channel to hold a nematode and that allows for movement, feeding and defecation.

In embodiments, the microfluidic device comprises at least one channel configured to hold one or more nematodes in fluid and may be any microfluidic chip known to one skilled in the art. Other microfluidic features include an inlet port, an outlet port, and one or more reservoirs. In exemplary embodiments, the microfluidic chip is made from the silicone polymer polydimethylsiloxane (PDMS), e.g. ScreenChip 40 (NemaMetric Inc,). In embodiments, the microfluidic chip comprises one or more channels, each channel configured to hold one nematode in fluid.

In embodiments, a microfluidic device is made from an elastomeric material such as a silicone polymer (for example, poly(dimethyl siloxane) (PDMS)). Suitable PDMS polymers include, but are not limited to Sylgard® 182, Sylgard® 184, and Sylgard® 186 (Dow Corning, Midland, Mich.). In one non-limiting example, the PDMS is Sylgard® 184. Additional polymers that can be used to make the disclosed microinjection chip include acrylic, polyurethane, polyamides, polyethelyene, polycarbonates, polyacetylenes and polydiacetylenes, polyphosphazenes, polysiloxanes, polyolefins, polyesters (such as thermoset polyester (TPE)), polyethers, poly(ether ketones), poly(alkaline oxides), poly(ethylene terephthalate), poly(methyl methacrylate), polyurethane methacrylate (PUMA), polystyrene, thiol-enes, fluoropolymers (for example, perfluoropolyethers), Norland Optical Adhesive 81, and derivatives and block, random, radial, linear, or teleblock copolymers, cross-linkable materials such as proteinaceous materials and/or combinations of two or more thereof. Also suitable are polymers formed from monomeric alkylacrylates, alkylmethacrylates, alpha-methylstyrene, vinyl chloride and other halogen-containing monomers, maleic anhydride, acrylic acid, and acrylonitrile. Monomers can be used alone, or mixtures of different monomers can be used to form homopolymers and copolymers. See, e.g., U.S. Pat. No. 6,645,432; McDonald et al., Electrophoresis 21:27-30, 2000; Rolland et al., J. Am. Chem. Soc. 126:2322-2323, 2004; Carlborg et al., Lab Chip 11:3136-3147, 2011; Sollier et al., Lab Chip 11:3752-3765, 2011. In some examples, the channel of the device (such as a device made from PDMS) can be coated with a sol-gel. See Abate et al., Lab Chip 8:516-518, 2008, for example. In other embodiments, suitable materials for making the disclosed microfluidic device include polymeric films, photoresist, hydrogels, or thermoplastic polymers.

Microfluidic devices can be fabricated by methods known to one of ordinary skill in the art. In some examples the disclosed devices are made by molding uncured polymer from a photoresist master using standard photolithographic methods (e.g., U.S. Pat. No. 6,645,432; Madou, Fundamentals of Microfabrication, CRC Press, Boca Raton, Fla., 1997). In other examples, the disclosed devices are made by chemical etching, laser cutting, photopolymerization, lamination, embossing, or injection molding. In the case of glass devices, the microfluidic device can for instance be fabricated by etching the various types of channels into a thin glass plate and bonding this plate to a second glass plate that serves as a flat substrate. In an exemplary embodiment, the microfluidic device is fabricated according to methods disclosed in U.S. Pat. No. 9,723,817 or U.S. Ser. No. 16/449,438, herein incorporated by reference in its entirety. One of ordinary skill in the art can select an appropriate fabrication method based on the selected material for the device.

In embodiments, the microfluidic device comprises two or more electrodes, optionally integrated electrodes, wherein the electrodes can measure an electrical event of a nematode located in a microfluidic channel (also referred to herein as a recording channel). In certain embodiments, the microfluidic device comprises two or more integrated electrodes directly connected to the recording channel, and at least one differential amplifier or at least one voltage-clamp amplifier, wherein the amplifier is connected to an output from the two or more integrated electrodes.

In embodiments, the recording channel contains an electrically conductive buffer solution (such as a saline solution) which provides electrical continuity between electrodes and the nematodes. In embodiments, the buffer solution further comprises nematode food.

In embodiments, electrical contact with the recording channel is achieved by means of electrodes embedded in the material that forms the microfluidic device (such as an integrated electrode). Integrated electrodes can be included in any suitable material (for example, glass, PDMS, polycarbonate, acrylic, or other polymeric material). Integrated electrodes can be fabricated by any means that yields spatially patterned conductive elements that serve as wires. In one non-limiting example, the electrodes are composed of indium tin oxide. In another example, electrodes are composed of metallic silver. Patterning of electrode materials can be achieved for example, using photolithography combined with etching.

In some embodiments, the system includes the microfluidic device and one or more nematodes in a recording channel, two or more electrodes, one or more amplifiers, which are connected to outputs from each electrode, an oscilloscope, which receives input from the amplifier, a data acquisition unit, which receives input from the amplifier; and a computer, which receives input from the data acquisition unit. In some examples, the system also includes a means for regulating flow of solutions through the device (such as a pump, for example, a syringe pump). One of ordinary skill in the art can utilize the systems disclosed herein to measure electrical events of defecting nematodes. In embodiments the nematodes comprise C. elegans. In other embodiments, the nematodes comprise parasitic nematodes.

The nematodes are introduced to the microfluidic device by any convenient means. In some examples, the nematodes are introduced into the device by transferring the nematodes to an inlet port (which may be pre-loaded with an aqueous buffer) and applying gentle pressure (for example, from a syringe) to move the nematodes into a reservoir and the recording channel. In certain embodiments, the cuticle of the nematode is made more permeable to drugs and test compounds by means of chemical treatments and/or genetic mutations. In other embodiments, the ability of the nematode to capture and/or excrete foreign chemicals is compromised by genetic mutation of endogenous pumps and other proteins.

Electrical recordings are made using standard techniques known to one of ordinary skill in the art. In some examples, electrical events are recorded by AC differential amplifiers connected to metal electrodes integrated into the device. Signals are displayed on oscilloscopes and recorded for later analysis using a data acquisition system connected to a computer running data acquisition software. Data analysis is performed offline after experiments. Raw electrical event recordings can be filtered to remove slow drift and high-frequency noise. The power spectrum can be computed as function of time or experimental treatments, including drugs, mutants, and toxic compounds.

Nematodes

In embodiments, the nematodes are selected from C. elegans; parasitic nematodes; transgenic or variant nematodes; nematodes that express one or more human genes, or variants thereof; or, wild type nematodes.

In embodiments, the nematode is a transgenic nematode comprising a heterologous gene, wherein exon coding sequences of the heterologous gene comprises one or more mutations resulting in an amino acid change as compared to a wildtype reference sequence. Making transgenic organism, including nematodes, via genetic engineering in which DNA is inserted, replaced, or removed from a genome using gene editing tools, is well known in the art. Examples of gene editing tools include, without limitation, zinc finger nucleases, TALEN and CRISPR. In embodiments, a host nematode is a C. elegans, C. briggsae, C remanei, C. tropicalis, or P. pacificus. (Sugi T et al. Genome Editing in C. elegans and Other Nematode Species. Int J Mol Sci. 2016 Feb. 26; 17(3):295.

In embodiments, a host nematode comprises a heterologous gene, wherein the entire host nematode ortholog was removed, either prior to or at the same time the heterologous gene was installed, and wherein the heterologous gene is installed at the host nematode ortholog native locus. See U.S. patent Ser. No. 16/381,988, the contents of which are incorporated herein by reference. In embodiments, the heterologous gene is selected from a different species of nematode (e.g. parasitic nematode), an avian, mammal or fish. In embodiments, the heterologous gene is a human gene, including variants thereof. In certain embodiments, the human gene is a clinical variant.

In embodiments, a heterologous gene replaces the entire nematode ortholog gene at the native locus, accordingly the heterologous gene must have a homolog as an identified ortholog in the host nematode. In one embodiment, the homolog is of substantial quality when sequence identity between heterolog source and host exceeds 70%. In one embodiment, the homolog is of high quality when sequence identity between heterolog source and host exceeds 50%. In other embodiments, the homolog is good when its identity exceeds 35%. In other embodiments, the homolog is adequate when its identity exceeds 20%. In other embodiments, the homolog is poor but acceptable when its identity is less than 20%.

In alternative embodiments, the heterologous gene is from a parasitic nematode, which are selected from Trichuris muris, Ascaris lumbricoides, Ancylostoma duodenale, Necator americanus, Trichuris trichiura, Enterobius vermicularis, Strongyloides stercoralis, Trichinella spiralis, Wuchereria bancrofti, Brugia malayi, Brugia timori, Loa loa, Mansonella streptocerca, Onchocerca volvulus, Mansonella perstans, Mansonella ozzardi, Cooperia punctata, Cooperia oncophora, Ostertagia ostertagi, Haemonchus contortus, Ascaris suum, Aphelenchoides, Ditylenchus, Globodera, Heterodera, Longidorus, Meloidogyne, Nacobbus, Pratylenchus, Trichodorus, Xiphinema, Bursaphelenchus, Dirofilaria immitis, Toxocara canis, Toxocara cati, Ancylostoma braziliense, Ancylostoma tubaeforme, Ancylostoma caninum, Dirofilaria repens, and Uncinaria stenocephala.

In certain embodiments, the heterologous gene is a human gene. In certain embodiments, the human gene is a wild type gene. Provided herein is a transgenic nematode system comprising a host nematode comprising a heterologous gene optimized for expression in the host nematode wherein the heterologous gene replaced a host nematode gene ortholog and the heterologous gene rescues, or at least partially restores, function of the replaced nematode ortholog. Heterologous genes that rescue function of the replaced nematode ortholog are referred to herein as “wild type” heterologous genes.

In other embodiments, the heterologous gene is a human disease gene. As used herein, “disease gene” refers to a gene involved in or implicated in a disease. In certain embodiments provided herein are transgenic nematodes comprising a heterologous gene that is a human wild type disease gene that has replaced the host nematode ortholog at the native locus. Those human heterologous disease genes represent targets for drug discovery and drugs that rescue function of human clinical variants.

The present transgenic nematodes may be prepared via homologous recombination at the native locus of the host nematode ortholog wherein the nematode ortholog is replaced with the heterologous gene. This method is advantageous in that it provides a platform for further testing and modifications and provides an improvement over previously disclosed methods that use amino acid substitution for generation of humanized nematodes expressing clinical variants. The use of gene-swap (i.e. heterologous gene replaces the nematode ortholog at the native locus) avoids the expression level issues that are a challenging problem with extrachromosomal array studies. Instead, CRISPR techniques are deployed to directly mutate at native loci. Farboud B and Meyer BJ. Dramatic enhancement of genome editing by CRISPR/Cas9 through improved guide RNA design. Genetics. 2015 April; 199(4):959-71; Paix A et al. High Efficiency, Homology-Directed Genome Editing in Caenorhabditis elegans Using CRISPR-Cas9 Ribonucleoprotein Complexes. Genetics. 2015 September; 201(1):47-54.

In certain embodiments, the transgenic nematodes may be prepared by methods other than homologous recombination into the native locus of the nematode, provided the cDNA of the heterologous gene is optimized for expression in the host nematode by codon optimization, addition of host intron sequences to the cDNA sequence of the heterologous gene and removing aberrant splice donor and acceptor sites. Those alternative methods comprise inserting the optimized heterologous gene via homologous recombination into a native locus of the nematode wherein a nematode gene ortholog is removed, wherein the heterologous gene rescued, or at least partially restored, function of the removed nematode ortholog; or, inserting the optimized heterologous gene into a non-native locus of the nematode; or, inserting the optimized heterologous gene into a random site of the nematode genome; or, adding the optimized heterologous gene as an expression vector wherein the optimized heterologous gene is not integrated into the nematode genome.

In embodiments are provided transgenic nematodes comprising a variant of the heterologous gene. As used herein, “variant heterologous gene” refers to an expressed gene with one or more amino acid changes as compared to the heterologous wild type gene. The transgenic nematodes comprising a variant heterologous gene may be used for assessing function of the heterologous variant gene (e.g. functional analysis) and drug discovery.

In embodiments, the variant heterologous gene is a human disease gene comprising one or more amino acid changes as compared to the wild type disease gene. In embodiments, the variant comprises a single amino acid change wherein the change was installed into the integrated heterologous sequence of the transgenic animal via a co-CRIPSR method. In certain embodiments, the mutations (of the heterologous exon coding sequence) are created from a pool of DNA repair templates each containing one or more mutations. In other embodiments, the variant comprises more than one amino acid change. In certain embodiments, those mutations are created from a pool of DNA repair templates each containing two or more mutations. Variants with more than one amino acid change, as compared to the wild type gene, may be a known clinical variant or a combination of two or more variants of the same gene. The combination of clinical variants in one variant heterologous transgenic nematode may be beneficial for assessing function of variants as to their synergistic, antagonistic, additive etc. function as measured in the present defecation phenotype methods.

In embodiments, the variant heterologous gene is a human clinical variant. Six classes of clinical variants can be installed (Pathogenic, Likely Pathogenic, Uncertain Significance, Likely Benign, Benign, and the unassessed). On average, dbSNP data indicates 80% of known variants are unassessed and nearly half (40%) of the remaining assessed variants are Variants of Uncertain Significance (VUS). (NCBI) Variation Viewer. Installation of known Pathogenic and Benign variants helps determine how conserved the existing assignments are when installed into the human cDNA expressing nematode model.

In embodiments, methods are provided herein for assessing function of a human clinical variant, comprising the steps of culturing a transgenic nematode, wherein the variant heterologous gene is a human clinical variant; and, performing a defecation motor assay using a microfluidic device, wherein a change in phenotype as compared to a control transgenic nematode comprising a wildtype heterologous gene indicates an altered function of the clinical variant in the transgenic nematode. In embodiments, the methods further comprise classifying the human clinical variant as pathogenic, likely pathogenic, uncertain significance, likely benign, or benign following the phenotypic screen.

In further embodiments provided herein are methods using the transgenic nematode system for drug screening. For humanized platforms exhibiting pathogenic activity with a given installed variant, screens of novel and existing compounds can be performed in efforts to find drug candidates with capacity to restore function back towards wild type. In embodiments, the methods for screening therapeutic agents to treat altered function of a human clinical variant, comprises placing a test transgenic nematode in a medium comprising a test compound, wherein the variant heterologous gene is a human clinical variant identified as pathogenic, likely pathogenic, unknown significance or unassigned; incubating the test transgenic nematode with the test compound for a period from 2 minutes to 7 hours; and, performing the instant defection assay in a microfluidic device, whereby therapeutic agents are identified from the test compounds when the outcome of the screening assay is deemed positive. An altered phenotype back towards wildtype is conserved positive.

Methods

Provided herein are methods for detecting and measuring defecation motor program (DMP) events of nematodes using a microfluidic device. In certain embodiments, the methods comprise use of a fluorescent or colored material that is ingested by the nematodes and a fluorescent microscope to visualize the events wherein the nematodes are maintained in fluid filled channels of the microfluidic device. The three distinct steps of the defection motor program are visualized using this method. In certain other embodiments, the methods use a microfluidic device comprising at least two electrodes wherein the nematodes are placed in a microfluidic/recording channel in an aqueous buffer comprising nematode food and defecation events are measured as an electrical event of the expulsion muscle contraction, which is distinguished from electrical events of feeding. See FIG. 2. As disclosed in the Background section, defecation is carried out by three distinct motor steps: the posterior body muscle contraction (pBoc), the anterior body muscle contraction (aBoc), and the expulsion muscle contraction (EMC). Together, these steps constitute the defecation motor program (DMP).

In embodiments, any fluorescent or colored material that can be ingested by a nematode may be used in the present methods. In embodiments, the fluorescent or colored material is bacterial feed that comprises a fluorescent dye, color dye, or an expressed fluorescent protein. In exemplary embodiments, the fluorescent or colored material is bacteria mixed with and comprising a fluorescent dye. In alternative embodiments, the nematodes are induced to feed, such as with the dopamine, wherein nanoparticles comprising a fluorescent dye or color dye may be used. In that instance, nematodes are stimulated to feed using dopamine and ingest nanoparticles, such as beads encapsulating a fluorescent dye or color dye. Nematodes feed until their gut is filled with the fluorescent or colored material and then transferred to a microfluidic device.

When placed in a channel of the microfluidic channel, the at least one nematode is optionally stimulated to feed and defecate wherein events associated with DMP may be observed and recorded. In embodiments, the nematodes are stimulated with dopamine or serotonin. In other embodiments, the nematodes are stimulated to feed and defecate with the presence of bacterial feed. In certain embodiments, the microfluidic device is placed on a stage of a fluorescent microscope, wherein an excitation and emission filter are selected depending on the fluorophore used in the present methods. One of skill in the art understands the fluorescent microscopes that may be used in the present methods along with the appropriate filters to allow observation, detection and/or recording of DMP events using a fluorescent or color material that was ingested by the nematodes, passed through the gut, and subsequently excreted. In other certain embodiments, the microfluidic device is used to measure electrical events associated with the nematode DMP.

Provided herein is a system for performing a nematode defecation motor assay using a microfluidic chip. In embodiments, the system comprises a microfluidic chip comprising at least one channel configured to hold individual nematodes in each channel, at least one nematode comprising ingested fluorescent or colored material and placed in the at least one channel of the microfluidic chip, and a fluorescent microscope with excitation and emission filters selected for a fluorophore or chromophore of the ingested fluorescent or colored material.

In certain embodiments, the system includes the microfluidic device, a fluorescent microscope, a data acquisition unit, which receives input from the microscope; and a computer, which receives input from the data acquisition unit. In some examples, the system also includes a means for regulating flow of aqueous solutions through the microfluidic device (such as a pump, for example, a syringe pump). One of ordinary skill in the art can utilize the systems disclosed herein to observe and measure DMP events of at least one nematode. In embodiments the nematodes comprise C. elegans. In other embodiments, the nematodes comprise parasitic nematodes. In embodiments, the nematodes comprise: C. elegans; parasitic nematodes; transgenic or variant nematodes; nematodes that express one or more human genes; or, wild type nematodes.

The nematodes are introduced into the microfluidic device by any convenient means. some examples, the nematodes are introduced into the device by transferring the nematodes to an inlet port (which may pre-loaded with an aqueous buffer solution) and applying gentle pressure (for example, from a syringe) to move the nematodes into a reservoir and/or a channel. In certain embodiments, the cuticle of the nematode is made more permeable to drugs and test compounds by means of chemical treatments and/or genetic mutations. In other embodiments, the ability of the nematode to capture and/or excrete food and/or foreign chemicals is compromised by genetic mutation of endogenous pumps and other proteins.

In certain embodiments, are methods for performing functional analysis of a genetic variant, comprising providing a transgenic nematode comprising a heterologous gene, wherein exon coding sequences of the heterologous gene comprises one or more mutations resulting in an amino acid change as compared to a wildtype reference sequence; introducing the transgenic nematode into a microfluidic device configured to hold the nematode in a microfluidic channel; performing a nematode defecation motor assay using a microfluidic device; comparing results of the defecation motor assay to a result from a defecation motor assay of a control nematode to identify a change between results, whereby functional analysis of a genetic variant is performed.

Disclosed herein are methods for screening test compounds by observing, measuring and/or recording DMP events from at least one nematode in comparison to a control. In embodiments, the method comprises contacting the at least one nematode with the test compound (before, after or simultaneously with ingestion of food), observing, measuring and/or recording DMP events of the contacted at least one nematode, comparing the recorded DMP events to a control DMP event, and determining if the recorded DMP event is altered as compared to the control DMP event, whereby test compounds are screened.

In embodiments, the test compound is selected from a drug, a drug candidate, an industrial chemical, or an environmental pollutant. In certain embodiments, the drug or drug candidate is selected from an organic compound, an inorganic compound, a hormone, a growth factor, a cytokine, a receptor, an antibody, an enzyme, a peptide, an aptamer or a vaccine.

In certain embodiments, the disclosed methods include screening for anthelmintic or antimicrobial compounds. In other embodiments, the methods include screening for compounds of use for treating neuromuscular diseases (such as muscular dystrophies, for example, Duchenne muscular dystrophy), neurodegenerative diseases (such as Alzheimer disease, Parkinson disease, Huntington disease, or tauopathies), mitochondrial disorders, or substance abuse disorders.

Methods of screening for or identifying anthelmintic compounds include introducing nematodes (such as C. elegans) in a microfluidic device disclosed herein, contacting the nematode with one or more test compounds, and recording DMP events from the nematodes, as disclosed above. The DMP event in the presence of the one or more test compounds is compared to a control (such as DMP events from the same or a different C. elegans in the absence of the test compounds) and the compound is identified as an anthelmintic or candidate anthelmintic if the DMP event is altered in the presence of the test compound as compared to the control. In embodiments, the nematodes are contacted with dopamine, serotonin or bacterial food prior to and/or concurrent with the test compound to stimulate feeding and defecation.

Methods of screening for or identifying compounds of potential use for treating disease, such as neurodegenerative disease (for example, Parkinson disease, Huntington disease, Alzheimer disease), neuromuscular disease (for example, spinal muscular atrophies or amyotrophic lateral sclerosis), and muscular degenerative disease (for example, muscular dystrophies or sarcopenia) and/or inhibiting or reducing aging include introducing nematodes (such as C. elegans) in a microfluidic device, contacting at least one nematode with one or more test compounds, and recording DMP events from the nematodes, as disclosed above. In certain embodiments, such as diseases for which the C. elegans genome contains a gene that is orthologous to the human gene implicated in the disease, a strain is created or obtained in which that gene is mutated and is utilized in the screening methods. In embodiments, a strain is created in which the human gene is expressed in C. elegans by transgenic techniques. Strains that are disease models can be used in drug screens by searching for compounds that mitigate one or more phenotypes in C. elegans. This mitigation can be the result of either chronic or acute exposure to a test compound. In one embodiment, recording DMP events from the at least one nematode is used to test for mitigation of disease phenotypes consisting of alterations in the behavior, physiology, and/or other aspects of posterior body muscle contraction (pBoc), anterior body muscle contraction (aBoc), and/or expulsion muscle contraction (EMC).

The test compounds used in the present invention include, but are not limited to drugs, drug candidates, biologicals, food components, herb or plant components, proteins, peptides, oligonucleotides, DNA and RNA. In embodiments, the test compound is a drug, a drug candidate, an industrial chemical, an environmental pollutant, a pesticide, an insecticide, a biological chemical, a vaccine preparation, a cytotoxic chemical, a mutagen, a hormone, an inhibitory compound, a chemotherapeutic agent or a chemical. In certain embodiments, the drug or drug candidate is selected from the group consisting of an organic compound, an inorganic compound, a hormone, a growth factor, a cytokine, a reception, an antibody, an enzyme, a peptide, an aptamer or a vaccine. The test compound can be either naturally-occurring or synthetic and can be organic or inorganic. A person skilled in the art will recognize that the test compound can be added to the at least one nematode and/or the microfluidic device in an appropriate solvent or buffer.

In embodiments, the test compound includes pharmacologically active drugs or drug candidates and genetically active molecules. Test compounds of interest include chemotherapeutic agents, anti-inflammatory agents, hormones or hormone antagonists, ion channel modifiers, and neuroactive agents. Exemplary of pharmaceutical agents suitable for this invention are those described in “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

In embodiments, the test compound includes all of the classes of molecules disclosed herein and may further or separately comprise samples of unknown content. While many samples will comprise compounds in solution, solid samples that can be dissolved in a suitable solvent may also be assayed. Samples containing test compounds of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include test compounds being assessed for potential therapeutic value, e.g., drug candidates from plant or fungal cells.

Test compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Use of a Microfluidic Device to Perform a Defecation Motor Assay

Provided herein is a method for performing a defection motor assay using a fluorophore to measure and record contraction and expulsion events of a nematode when the nematode is in a microfluidic channel of a microfluidic chip.

To visualize defecation in C. elegans, the nematodes were fed 1,1′-dioctadecyl-3,3,3′3′-tetramethylindocarbocyanine perchlorate (DiI) at a concentration of 2 μL diluted in a liquid culture of the OP50 strain of E. coli bacteria. 300 μL of the mixture was then pipetted onto a petri plate filled with nematode growth medium (NGM) agar. Those plates were then protected from light to preserve the activity of the fluorescent DiI dye and used within 48 hours.

Synchronized populations of nematodes were obtained by dissolving gravid hermaphrodites in a bleach solution. See Table 1. The nematodes were transferred into a microcentrifuge tube and submerged in the bleach solution. Once the hermaphrodite bodies were fully disintegrated, the embryos, which are protected from bleach by a chitinous shell, were spun down and the supernatant bleach removed. The pellet of eggs was resuspended and rinsed with sterile water. After the final rinse, the embryos were placed in a scintillation vial with M9 buffer. See Table 2. Twelve hours later the nematodes hatched and transferred to a nematode growth media (NGM) plate cultured with OP50; the plate placed in a 20° C. incubator wherein the nematodes developed to larval stage 4 (L4).

TABLE 1
Bleaching Solution

| Constituent | Amount (mL) |
|---|---|
| Distilled water | 3.675 |
| Sodium hypochlorite solution (5%) | 1.200 |
| NaOH solution (10M) | 0.125 |

TABLE 2
M9 Buffer Solution

| Constituent | Amount | |
|---|---|---|
| KH₂PO₄ | 3 | g |
| Na₂HPO₄ | 6 | g |
| NaCl | 5 | g |
| MgSO₄ (1M) | 1 | mL |
| Distilled water | to 1 | L |

Approximately 15-20 synchronized L4 larvae nematodes were rinsed from the plate they were grown on and placed onto NGM plates that were seeded with the DiI OP50 mixture. The plates were protected from light until the day of the assay to preserve the activity of DiI dye. The nematodes fed on the DiI E. coli mixture for at least 12 hours prior to assay beginning wherein at the end of that time period the nematodes' guts were filled with fluorescent bacteria; the contractions and expulsions of the Defecation Motor Program (DMP) were clearly visible with a 10× objective on a fluorescent microscope.

To immobilize and easily observe the contractions and expulsions of the Defecation Motor Program (DMP), the above nematodes were inserted into a ScreenChip 40 (NemaMetrix). ideal for Day 1 adult C. elegans, with 20 mg/mL OP50 (LabTie Freeze Dried OP50) in M9 buffer. The ScreenChip SC40 consists of a rectangular block composed of polydimethylsiloxane (PDMS) measuring 45 mm×14 mm×5 mm bonded to a 1 mm thick glass microscope slide 75 mm×25 mm. Two fluidic features, connected in series, were molded into the bottom of the block: a reservoir which accepts a population of nematodes suspended in a buffer solution, and a 40 um wide recording channel. The reservoir included 1.0 mm diameter inlet and outlet ports. Three electrodes, consisting of indium tin oxide (ITO), are deposited on the slide, configured to make contact with any buffer solution filling the recording channel. Regions of the electrodes beneath the PDMS block had a high length to width ration to increase the fluidic resistance of these potential leakage pathways.

Prior to loading the nematodes in the microfluidic chip, M9 buffer was used to rinse approximately 5-10 animals off the DiI/OP50 mixture petri plate and transferred to a microcentrifuge tube wrapped in tinfoil to preserve the activity of DiI throughout the assay. The worms settled to the bottom of the tube and the M9 supernatant was discarded, leaving only the worm pellet. 100 μL of a liquid culture of the OP50 strain of E. coli at a concentration of 20 mg/mL was added to the tube. The ScreenChip 40 was placed in a dock for stability and ease of use. A syringe attached to a 4-inch piece of polyethylene tubing (1.4 mm ID, 1.9 mm OD), with a stainless-steel tube (1.2 mm ID, 1.47 mm OD, 12.7 mm long) at the terminus was inserted into the microcentrifuge tube containing the worms and the E. coli solution with the nematode mixture was suctioned into the syringe assembly. The stainless-steel tube was then inserted half-way into the open inlet port of the microfluidic device and positive pressure was applied to the syringe to push the nematodes into the microfluidic device. Light pressure was applied to the syringe until an individual nematode was secured in the recording channel of the microfluidic chip. The dock and chip were placed on the stage of a fluorescent microscope with a 457-493 nm excitation filter and 508-552 nm emission filter and imaged at 10×. magnification.

The nematodes adjusted to the recording channel of the device for 20-30 second before starting the assay. During the assay, individual nematodes were observed for 10 minutes. Expulsion events and intestinal contraction were easily observable as fluorescent material filled the intestines and left the soma of the worm. See FIG. 1.

### Example 2: Electrical Measurement of Defecation Phenotype

Provided herein is a method for performing a defecation motor assay using a microfluidic device with two or more electrodes connected to an amplifier to measure and record expulsion events of a nematode when the nematode is in a microfluidic channel of a microfluidic device.

In this example, nematodes were placed in a loosely fitting microfluidic chamber between a positive and a negative electrode connected to an amplifier. The expulsion from the gut of excreta was detected as an electrical event, representing an electrical muscle discharge. See FIG. 2. This electrical event can be detected regardless of the orientation of the nematode in the microfluidic channel, in other words whether the worm's head is nearest the positive or negative electrode.

The electrical event can be detected either by recording and amplifying a time-varying voltage signal or by recording a derived current signal resulting from passing a known time-varying voltage signal between the two electrodes and recording a change in the resistance. Nematodes used in these recordings can be from a synchronous (age and/or size) or asynchronous population.

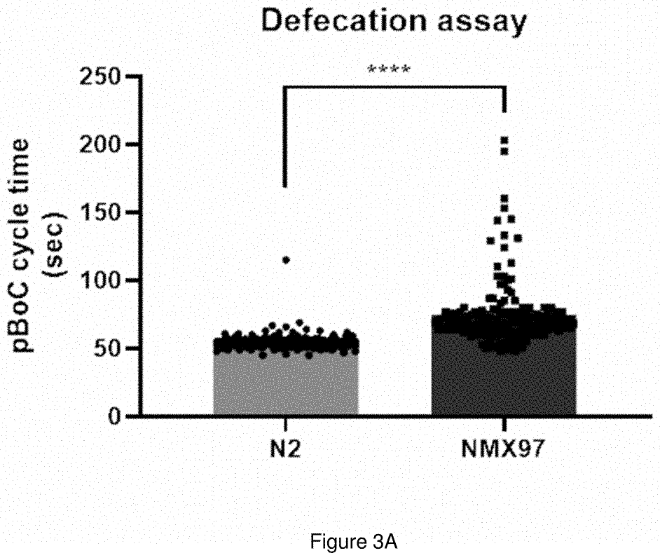

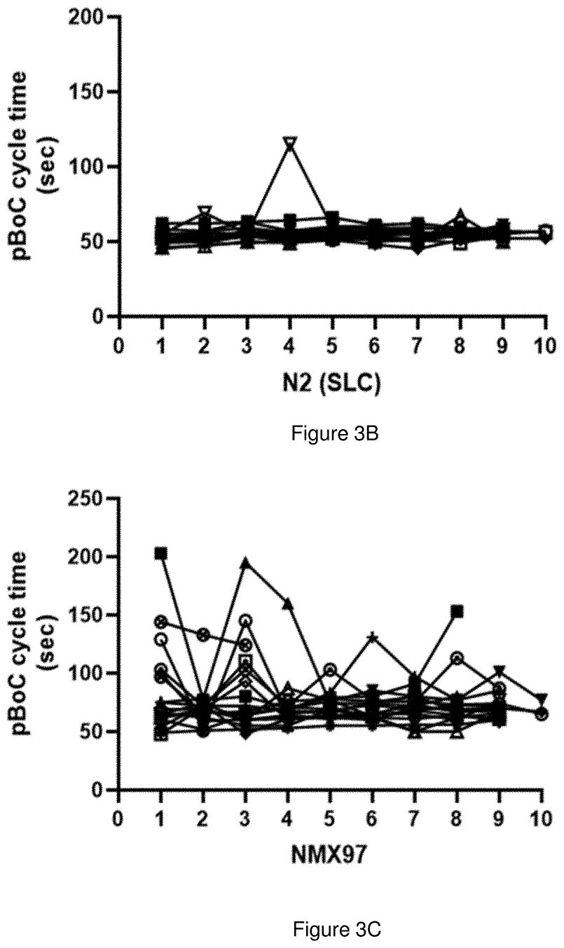

The phenotype of peristaltic defecation was used to distinguish genetic knockouts from wild type nematodes. The knockout of the ortholog of the human gene KCNQ2, kqt-3, results in a disruption of the peristaltic rhythm and an increase in the interval between successive defecation events. See FIGS. 3A to 3C. Nematodes of each genotype were measured for 10-20 minutes with food stimulation and after adaptation to detect defecation events and the interval between successive events. Ten to twenty nematodes of each genotype were recorded and analyzed. Both the mean interval between successive events and the interval between events was disrupted in the case of the genetic knockout.

These results indicate that loss of function mutants in variants of this gene either in the native coding sequence of kqt-3 or in a transgenic nematode with KCNQ2 installed at the orthologous kqt-3 locus would be likely to lead to a similar phenotype of lengthened periodicity between successive events. Gain-of-function mutations in the same gene may lead to opposite directionality of phenotype in the same assay, i.e., decreased length of time between successive events. These results, paired with knowledge about the protein produced by the genes coding sequence, can provide information about how a particular variant produces an aberrant pathological phenotype, i.e. the mechanism and mode of action.

Further, and more generally, defecation strength and timing phenotype or assay methods can be used alone or with other phenotype assays to perform functional analysis of genetic variants, prediction of whether a genetic change is pathogenic or benign to organisms, predict mechanism of action in phenotypic changes for the purposes of identifying drug targets and identifying new drugs, or for capturing effects of drug or compound application on a particular genetic variant representing a patient for the purposes of drug regimen discovery, drug screening, or drug repurposing.

1. A method of performing a nematode defecation motor assay using a microfluidic device, comprising:
a. inducing at least one nematode to ingest a fluorescent or colored material;
b. introducing the at least one nematode after ingesting the fluorescent or colored material into a microfluidic device configured to hold the nematode in a microfluidic channel; and,
c. placing the microfluidic device on a stage of a fluorescent or brightfield microscope and observing contraction and expulsion events of the at least one nematode, whereby the nematode defecation motor assay is performed.

2. The method of claim 1, wherein the at least one nematode is induced to ingest the fluorescent or colored material with the presence of bacterial feed.

3. The method of claim 1, wherein the at least one nematode is induced to ingest the fluorescent or colored material with serotonin.

4. The method of claim 1, wherein the fluorescent or colored material is bacterial feed comprising a fluorophore or chromophore.

5. The method of claim 4, wherein the fluorophore is a fluorescent dye or a fluorescent protein.

6. The method of claim 1, wherein the fluorescent or colored material is a nanoparticle comprising a fluorophore or chromophore.

7. The method of claim 1, wherein the microfluidic device comprises one or more channels, each channel configured to hold one nematode in fluid.

8. The method of claim 1, wherein microfluidic device comprises a silicone polymer, a thermoplastic polymer, an acrylic polymer, or a polycarbonate polymer.

9. The method of claim 8, wherein the thermoplastic polymer comprises poly(methyl methacrylate) (PMMA), polycarbonate (PC), polystyrene (PS), polyvinyl chloride (PVC), polyimide (PI), olefin polymers, cyclic olefin copolymer (COC), cyclic olefin polymer (COP), or cyclic block copolymer (CBC).

10. The method of claim 8, wherein the silicone polymer comprises a polydimethylsiloxane (PDMS) elastomer.

11. A system for performing a nematode defecation motor assay using a microfluidic device, comprising:
a. a microfluidic device comprising at least one channel configured to hold individual nematodes in each channel;
b. at least one nematode comprising ingested fluorescent or colored material and placed in the at least one channel of the microfluidic device; and,
c. a fluorescent microscope with excitation and emission filters selected for a fluorophore or chromophore of the ingested fluorescent or colored material.

12-14. (canceled)

15. A method of performing a nematode defecation motor assay using a microfluidic device measuring an electrical event of an electrical muscle discharge, comprising:
a. introducing the nematode into a microfluidic device configured to hold the nematode in a microfluidic channel, wherein the microfluidic device comprises two or more electrodes directly connected to the microfluidic channel; and,
b. measuring the electrical event of the nematode; and,
c. recording the electrical event as an electrical muscle discharge whereby the nematode defecation motor assay is performed.

16. The method of claim 15, wherein the nematode is present in an aqueous buffer solution comprising food.

17. The method of claim 15, wherein the nematode ingested food prior to introduction into the microfluidic device.

18-21. (canceled)

22. A method for performing functional analysis of a genetic variant using the system of claim 11, comprising:
a. providing a transgenic nematode comprising a heterologous gene, wherein exon coding sequences of the heterologous gene comprises one or more mutations resulting in an amino acid change as compared to a wildtype reference sequence;
b. introducing the transgenic nematode into a microfluidic device configured to hold the nematode in a microfluidic channel;
c. performing a nematode defecation motor assay using a microfluidic device;
d. comparing results of the defecation motor assay to a result from a defecation motor assay of a control nematode to identify a change between results, whereby functional analysis of a genetic variant is performed.

23. The method of claim 22, further comprising placing the microfluidic device on a stage of a fluorescent microscope and observing contraction and expulsion events of the transgenic nematode.

24-29. (canceled)

30. The method of claim 22, wherein the microfluidic device comprises two or more electrodes directly connected to the microfluidic channel, and the defection motor assay is performed by measuring the electrical event of the nematode and recording the electrical event as an electrical muscle discharge.

31. The method of claim 22, further comprising introducing a therapeutic agent into the microfluidic device with the transgenic nematode.

32. The method of claim 31, further comprising identifying therapeutic agents that alter the defecation motor phenotype of the transgenic nematode.

33. The method of claim 22, further comprising analyzing directionality of the change in results to determine mode of action of the genetic variant.
